(19) United States
(12) Patent Application Publication
(10) Pub. No.: US 2023/0074641 A1
(43) Pub. Date: Mar. 9, 2023

(54) BIOPHARMACEUTICAL MANUFACTURING PROCESS AND PRODUCT

(71) Applicant: SILCOTEK CORP., Bellefonte, PA (US)

(72) Inventors: Jesse BISCHOF, Bellefonte, PA (US); Martin E. HIGGINS, Bellefonte, PA (US); Thomas WINTER, Osage Beach, MO (US)

(21) Appl. No.: 17/795,081

(22) PCT Filed: Jan. 22, 2021
(86) PCT No.: PCT/US2021/014630

§ 371 Date: Jul. 25, 2022

Related U.S. Application Data
(60) Provisional application No. 62/966,276, filed on Jan. 27, 2020.

(51) Int. Cl. C23C 16/04; C23C 16/24; C23C 16/26; B01J 19/02; C09D 5/08; C09D 1/00

(52) U.S. Cl. CPC C23C 16/045; C23C 16/24; C23C 16/26; B01J 19/02; C09D 5/08; C09D 1/00; B01J 2219/0218; B01J 2219/0286

(57) ABSTRACT

Pharmaceutical manufacturing processes and products are disclosed. A pharmaceutical manufacturing process includes flowing a liquid through a pathway. The liquid contacts a non-polymeric coating on a substrate within the pathway. The substrate is a metal or metallic substrate. A pharmaceutical product is produced by flowing a liquid through a pathway. The liquid contacts a non-polymeric coating on a substrate within the pathway. The substrate is a metal or metallic substrate.

### PRIORITY

The present application is a non-provisional patent application claiming priority to and benefit of U.S. Provisional Patent Application No. 62/966,276, filed Jan. 27, 2020, and entitled “BIOPHARMACEUTICAL MANUFACTURING PROCESS AND PRODUCT,” the entirety of which is incorporated by reference.

### FIELD OF THE INVENTION

The present invention is directed to pharmaceutical manufacturing processes. More particularly, the present invention is directed to pharmaceutical manufacturing processes using a non-polymeric coating in contact with a liquid.

### BACKGROUND OF THE INVENTION

Pharmaceutical manufacturing processes are heavily scrutinized for safety and precision. Preventing accumulation of materials, inconsistent processing, or growth of bacteria are serious issues that must be prevented. To address such concerns, pharmaceutical manufacturers, along with regulatory entities and industry groups, have developed standards for hygiene.

Standards for hygiene such as those published in 2019 by the American Society of Mechanical Engineers (ASME) Bioprocessing Equipment (BPE) address some of these issues but have limitations. For example, the ASME-BPE standards limit materials in high-sensitivity environments to super alloys, 316L stainless steel, and specialized polymers. Such limitations prevent the use of non-polymeric coatings that have additional properties.

One example of an issue not fully resolved by ASME-BPE relates to guanidine hydrochloride. Guanidine hydrochloride (sometimes referred to as guanidinium chloride) is a powerful protein denaturing agent. In the pharmaceutical manufacturing processes, it is used as a cleaning agent in high concentrations. Concentrations of 6M or higher cause most proteins to lose their ordered structure and turn into randomly-coiled molecules. At such concentrations, it can corrosively attack stainless steel, so either stainless steel should not used in such situations or a lower concentration of guanidine hydrochloride should be used.

Pharmaceutical manufacturing processes and pharmaceutical products produced by such pharmaceutical manufacturing processes that show one or more improvements in comparison to the prior art would be desirable in the art.

### BRIEF DESCRIPTION OF THE INVENTION

In an embodiment, a pharmaceutical manufacturing process includes flowing a liquid through a pathway. The liquid contacts a non-polymeric coating on a substrate within the pathway. The substrate is a metal or metallic substrate.

In another embodiment, a pharmaceutical product is produced by flowing a liquid through a pathway. The liquid contacts a non-polymeric coating on a substrate within the pathway. The substrate is a metal or metallic substrate.

In another embodiment, a pharmaceutical manufacturing process includes flowing active pharmaceutical ingredients through a pathway having a weld. The active pharmaceutical ingredients contact a non-polymeric coating on a stainless steel substrate within the pathway (the non-polymeric coating including silicon, oxygen, carbon, and hydrogen) and produces a pharmaceutical product. The substrate is a metal or metallic substrate. The substrate is susceptible to corrosion from the active pharmaceutical ingredients and the non-polymeric coating does not corrode from the liquid.

Other features and advantages of the present invention will be apparent from the following more detailed description, taken in conjunction with the accompanying drawings which illustrate, by way of example, the principles of the invention.

### BRIEF DESCRIPTION OF THE DRAWINGS

FIG. 1 is a schematic perspective view of a portion of a pharmaceutical manufacturing process producing pharmaceutical products, according to embodiments of the disclosure.

Wherever possible, the same reference numbers will be used throughout the drawings to represent the same parts.

### DETAILED DESCRIPTION OF THE INVENTION

Provided are pharmaceutical manufacturing processes and pharmaceutical products produced by such pharmaceutical manufacturing processes that show one or more improvements in comparison to the prior art would be desirable in the art. Embodiments of the present disclosure, for example, in comparison to concepts failing to include one or more of the features disclosed herein, expand the capabilities of pharmaceutical manufacturing, permit increased precision of pharmaceutical manufacturing, permits increased accuracy of pharmaceutical manufacturing, permit decreased sizes of flow paths to be used in pharmaceutical manufacturing, permit reduced duration of cleaning for pharmaceutical manufacturing, permit bioinert and/or protein anti-stiction operation of pharmaceutical manufacturing, permit operations with fluids incompatible with certain ASME-BPE approved materials (as published in 2019) under certain conditions (for example, ammonia, chlorine (both wet and dry), HCl, nitrating acid (nitric and sulfuric combination), phosphoric acid, sodium and potassium hydroxide, bleach, and sulfuric acid), permits operation with fluids incompatible with polyether ether ketone (for example, benzene sulfonic acid, chlorine (both wet and dry), nitric acid, sulfuric acid, carbolic acid, ultraviolet light, methylene chloride, dimethylsulfate, tetrahydrofuran, and other organic solvents), permit pharmaceutical manufacturing with rougher substrates than ASME-BPE standards (as published in 2019) (for example, having an average Ra roughness of 0.38 micrometers in high-sensitivity environments), permit pharmaceutical manufacturing to be safely performed with elements not currently permitted by ASME-BPE standards (for example, threaded pipes instead of weld and polish or tri-clamps), permit pharmaceutical manufacturing to be performed at higher pressures, permit pharmaceutical manufacturing to be performed at higher pressures, permit pharmaceutical manufacturing to be performed at higher temperatures, permit pharmaceutical manufacturing to be performed more economically (for example, by not requiring as many cleaning cycles, not using as much cleaning materials, not having as many process disruptions, not using mechanical cleaning, not resurfacing or reprocessing portions, not remediating portions, or not having as much downtime), or permit a combinations thereof.

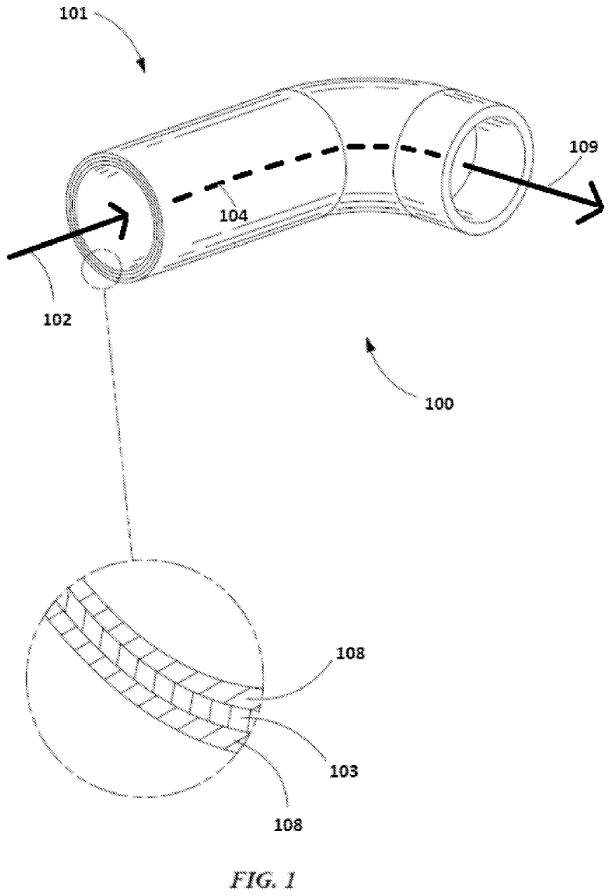

Referring to FIG. 1, a pharmaceutical manufacturing process 100 includes flowing a liquid 102 through a pathway 104. The liquid 102 contacts a non-polymeric coating 108 on a substrate 103 within the pathway 104. The substrate 103 is a metal substrate, a metallic substrate, a glass substrate, or a ceramic substrate. In further embodiments, a pharmaceutical product 109 is produced by the pharmaceutical manufacturing process 100.

The liquid 102 that contacts the non-polymeric coating 108 is or includes any liquid constituents of the pharmaceutical product 109, cleaning/sterilization solutions, or a combination thereof. Constituents of the pharmaceutical product 109 include, but are not limited to, water for injection, pure water, ultra-pure water, steam, treated potable water, untreated potable water, high-oxygen water (for example, greater than 90 mg/mL, 95 mg/mL, or 100 mg/mL, between 95 mg/mL and 105 mg/mL, between 99 mg/mL and 101 mg/mL, or any suitable combination, sub-combination, range, or sub-range therein), acids (for example, with a pH less than 7, between 5 and 7, between 3 and 5, between 1 and 3, or any suitable combination, sub-combination, range, or sub-range therein), bases (for example, with a pH greater than 7, between 7 and 9, between 9 and 11, greater than 11, or any suitable combination, sub-combination, range, or sub-range therein), buffers, process fluids, chorine, electropolishing fluids, cleaning/sterilization solutions, solvents, surfactants, oxidizers, phosphoric acid, guanidine hydrochloride, nitric acid, chelating agents, sanitizers, conditioners, bicarbonates, neutralizers, metal ions, hydroxides, particulate, contaminants, defect materials, or any suitable combination thereof. In one embodiment, the pharmaceutical product 109 is devoid or substantially devoid of one or more of the constituents.

The substrate 103 is a metal or metallic substrate that, in one embodiment, is susceptible to corrosion from the liquid 102. In one embodiment, the substrate 103 is or includes stainless steel, for example, 304 stainless steel, 316 stainless steel, 316L stainless steel, ferrous-based alloys, non-ferrous-based alloys, nickel-based alloys, high-nickel alloys (for example, Hastelloy C-276 or Hastelloy C22), stainless steels (martensitic or austenitic), aluminum alloys, high molybdenum steel alloys (for example, having molybdenum at concentrations greater than 304, 316, and 316L), composite metals, or combinations thereof. In one embodiment, the substrate 103 is the metallic material and is tempered or non-tempered, has grain structures that are equiaxed, directionally-solidified, and/or single crystal, has amorphous or crystalline structures, is a foil, fiber, a cladding, and/or a film.

In an alternative embodiment, the metallic material is replaced with a non-metallic material. Suitable non-metal or non-metallic materials include, but are not limited to, ceramics, glass, ceramic matrix composites, or a combination thereof.

The substrate 103 has any suitable roughness for the pharmaceutical manufacturing process 100. Suitable roughnesses include, but are not limited to, Ra of between 0.38 micrometers and 0.51 micrometers, Ra of greater than 0.51 micrometers, or any suitable combination, sub-combination, range, or sub-range therein.

The pathway 104 has geometric features capable of being coated through starved reactor thermal chemical vapor deposition, which allows three-dimensional non-line-of-sight coating. Suitable features include, but are not limited to, welds (for example, tack welds, arc welds, laser welds, or other high energy welds and/or low quality welds), solders, brazes, corners, threaded portions, notches, fittings (for example, unions, connectors, adaptors, other connections between two or more pieces of tubing, for example, capable of making a leak-free or substantially leak-free seal), compression fittings (including ferrules, such as, a front and back ferrule), tubing (for example, coiled tubing, tubing sections such as used to connect a sampling apparatus, pre-bent tubing, straight tubing, loose wound tubing, tightly bound tubing, and/or flexible tubing, whether consisting of the interior being treated or including the interior and the exterior being treated), valves (such as, gas sampling, liquid sampling, transfer, shut-off, or check valves, for example, including a rupture disc, stem, poppet, rotor, multi-position configuration, able to handle vacuum or pressure, a handle or stem for a knob, ball-stem features, ball valve features, check valve features, springs, multiple bodies, seals, needle valve features, packing washers, and/or stems), quick-connects, regulators and/or flow-controllers (for example, including o-rings, seals, and/or diaphragms), injection ports (for example, for gas chromatographs), in-line filters (for example, having springs, sintered metal filters, mesh screens, housings, and/or weldments), glass liners, probes, drilled and/or machined block components, manifolds, sealing hardware, reactors, process fluid containers, pump impellers, water system components (for treated or untreated water), or a combination thereof.

As used herein, the term non-polymeric, as it applies to the non-polymeric coating 108, refers to not having repeated carbon units present within polymers. Examples of such polymers include, but are not limited to, polytetrafluoroethylene, polyethylene, polypropylene, polystyrene, polyvinyl chloride, and polyacrylonitrile.

In one embodiment, the non-polymeric coating 108 includes silicon, oxygen, carbon, and hydrogen. In a further embodiment, the non-polymeric coating 108 further includes nitrogen. Any other suitable compositional constituents that prevent corrosion are present. For example, the suitable embodiments include the non-polymeric coating 108 being a silicon-containing coating, an amorphous silicon coating, an amorphous carbon-containing coating, a carbon-functionalized coating, or any suitable combination thereof.

In one embodiment, the coating 108 is formed by one or more of the following fluids being used as a precursor for chemical vapor deposition: silane, silane and ethylene, silane and an oxidizer, dimethylsilane, dimethylsilane and an oxidizer, trimethylsilane, trimethylsilane and an oxidizer, dialkylsilyl dihydride, alkylsilyl trihydride, non-pyrophoric species (for example, dialkylsilyl dihydride and/or alkylsilyl trihydride), thermally-reacted material (for example, carbosilane and/or carboxysilane, such as, amorphous carbosilane and/or amorphous carboxysilane), species capable of a recombination of carbosilyl (disilyl or trisilyl fragments), methyltrimethoxysilane, methyltriethoxysilane, dimethyldimethoxysilane, dimethyldiethoxysilane, trimethylmethoxysilane, trimethylethoxysilane, ammonia, hydrazine, trisilylamine, Bis(tertiary-butylamino)silane, 1,2-bis(dimethylamino)tetramethyldisilane, dichlorosilane, hexachlorodisilane), organofluorotrialkoxysilane, organofluorosilylhydride, organofluoro silyl, fluorinated alkoxysilane, fluoroalkylsilane, fluorosilane, tridecafluoro 1,1,2,2-tetrahydrooctylsilane, (tridecafluoro-1,1,2,2-tetrahydrooctyl) triethoxysilane, triethoxy (3,3,4,4,5,5,6,6,7,7,8,8,8-tridecafluoro-1-octyl) silane, (perfluorohexylethyl) triethoxysilane, silane (3,3,4,4,5,5,6,6,7,7,8,8,9,9,10,10,10-heptadecafluorodecyl) trimethoxy-, or a combination thereof.

Suitable concentrations of thermally-reactive gas used in the process 100, by volume, are between 10% and 20%, between 10% and 15%, between 12% and 14%, between 10% and 100%, between 30% and 70%, between 50% and 80%, between 70% and 100%, between 80% and 90%, between 84% and 86%, or any suitable combination, sub-combination, range, or sub-range therein.

### Examples

In a first example, a comparative example, a 316L stainless steel coupon is immersed within 6M guanidine hydrochloride to replicate a comparative pharmaceutical manufacturing process. After one week, the 316L stainless steel coupon is removed from the 6M guanidine hydrochloride, rinsed with deionized water, sonicated with deionized water for 15 minutes, and dried for one hour at 230° F. The 316L stainless steel coupon shows oxidation and rust (also known as rouging).

The 316L stainless steel coupon is again immersed in the 6M guanidine hydrochloride. After one month, the 316L stainless steel coupon is removed from the 6M guanidine hydrochloride, rinsed with deionized water, sonicated with deionized water for 15 minutes, and dried for one hour at 230° F. The rusting is more severe than the one-week exposure. In addition, the 6M guanidine hydrochloride shows a yellow tint, believed to be iron leaching out of the 316L stainless steel coupon into the solution.

In a second example, a coated coupon having the non-polymeric coating 108 according to the disclosure is immersed within 6M guanidine hydrochloride to replicate an embodiment of the pharmaceutical manufacturing process 100. After one week, the coated coupon having the non-polymeric coating 108 is removed from the 6M guanidine hydrochloride, rinsed with deionized water, sonicated with deionized water for 15 minutes, and dried for one hour at 230° F. The coated coupon is unaffected by the 6M guanidine hydrochloride.

The coated coupon is again immersed in the 6M guanidine hydrochloride. After one month, the coated coupon is rinsed with deionized water, sonicated with deionized water for 15 minutes, and dried for one hour at 230° F. The coated coupon remains unaffected by the 6M guanidine hydrochloride.

While the invention has been described with reference to one or more embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. In addition, all numerical values identified in the detailed description shall be interpreted as though the precise and approximate values are both expressly identified.

What is claimed is:

1. A pharmaceutical manufacturing process, comprising:
flowing a liquid through a pathway;
wherein the liquid contacts a non-polymeric coating on a substrate within the pathway;
wherein the substrate is a metal or metallic substrate.

2. The process of claim 1, wherein the liquid includes active pharmaceutical ingredients.

3. The process of claim 1, wherein the liquid includes water for injection.

4. The process of claim 1, wherein the liquid is an acid.

5. The process of claim 1, wherein the liquid includes phosphoric acid.

6. The process of claim 1, wherein the liquid includes guanidine hydrochloride.

7. The process of claim 1, wherein the liquid includes nitric acid.

8. The process of claim 1, wherein the substrate is susceptible to corrosion from the liquid and the non-polymeric coating does not corrode from the liquid.

9. The process of claim 1, wherein the substrate is stainless steel.

10. The process of claim 1, wherein the pathway includes a weld.

11. The process of claim 1, wherein the pathway includes a corner.

12. The process of claim 1, wherein the pathway includes a flange.

13. The process of claim 1, wherein the pathway includes threaded portions.

14. The process of claim 1, wherein the substrate has a surface roughness of between 0.38 micrometers and 0.51 micrometers.

15. The process of claim 1, wherein the substrate has a surface roughness of greater than 0.51 micrometers.

16. The process of claim 1, wherein the non-polymeric coating is a material outside of the scope of standards of the American Society of Mechanical Engineers (ASME) Bioprocessing Equipment (BPE) as published in 2019.

17. The process of claim 1, wherein the non-polymeric coating includes silicon, oxygen, carbon, and hydrogen.

18. The process of claim 1, wherein the non-polymeric coating includes silicon, nitrogen, oxygen, carbon, and hydrogen.

19. A pharmaceutical product, wherein the pharmaceutical product is produced by the process of claim 1.

20. A pharmaceutical manufacturing process, comprising:
flowing active pharmaceutical ingredients through a pathway having a weld, wherein the active pharmaceutical ingredient contacts a non-polymeric coating on a stainless steel substrate within the pathway, the non-polymeric coating including silicon, oxygen, carbon, and hydrogen;
producing a pharmaceutical product;
wherein the substrate is a metal or metallic substrate;
wherein the substrate is susceptible to corrosion from the active pharmaceutical ingredients and the non-polymeric coating does not corrode from the liquid.
